(19) United States
(12) Patent Application Publication
(10) Pub. No.: US 2013/0023773 A1
(43) Pub. Date: Jan. 24, 2013

(54) APPARATUS AND TECHNIQUES OF NON-INVASIVE ANALYSIS

(71) Applicants: Sanjay Krishna, Albuquerque, NM (US); Sanchita Krishna, Albuquerque, NM (US)

(72) Inventors: Sanjay Krishna, Albuquerque, NM (US); Sanchita Krishna, Albuquerque, NM (US)

(73) Assignee: STC. UNM, Albuquerque, NM (US)

(21) Appl. No.: 13/639,818

(22) PCT Filed: Apr. 7, 2011
(86) PCT No.: PCT/US11/31529

§ 371 Date: Oct. 5, 2012

Related U.S. Application Data
(60) Provisional application No. 61/372,625, filed on Aug. 11, 2010.
(60) Provisional application No. 61/321,581, filed on Apr. 7, 2010.

(51) Int. Cl. A61B 6/00

(52) U.S. Cl. 600/474

(57) ABSTRACT

Apparatus and methods, which comprise examination of an abnormality on a subject using a temperature stimulus applied to the subject, provide a non-invasive analysis technique. In an embodiment, a non-invasive infrared imaging technique can be used to observe the temporal response of a lesion to temperature stimuli to form a basis for evaluating the abnormality. A technique including applying temperature stimuli and detecting responses to the applied temperature stimuli provide a non-invasive technique that can be used to identify an abnormality on a subject and/or characteristics of the abnormality. In an embodiment, a non-invasive transient infrared imaging technique can be used to observe the temporal response of a lesion to temperature stimuli to form a basis for determining characteristics correlated to the lesion. Additional apparatus, systems, and methods are disclosed.

### CLAIM OF PRIORITY

This application claims the priority benefit of U.S. Provisional Application Ser. No. 61/321,581, filed 7 Apr. 2010, entitled “NON-INVASIVE TECHNIQUE FOR ANALYSIS OF ABNORMALITIES” and U.S. Provisional Application Ser. No. 61/372,625, filed 11 Aug. 2010, entitled “NON-INVASIVE TECHNIQUE FOR MEASUREMENT OF THICKNESS OF ABNORMALITIES,” which applications are each incorporated herein by reference in their entirety.

### FIELD OF THE INVENTION

This invention relates generally to the field of non-invasive diagnostics.

### BACKGROUND

Skin cancer is the most common cancer diagnosed in the United States, affecting more than 1 million Americans every year. In the United States alone, observed incidence increased by 126% between 1973 and 1995, at a rate of approximately 6% per year. Interestingly, there are more cases of skin cancer than there are of breast cancer, prostate cancer, lung cancer and colon cancer combined. It is alarming to note that one in every five Americans develops skin cancer in their lifetime.

Skin cancers are usually divided into (a) basal cell carcinoma (BCC), (b) squamous cell carcinoma (SCC) and (c) melanoma. Basal cell carcinoma is the most common form of skin cancer. It is rarely fatal but can be highly disfiguring. The deadliest form of skin cancer is melanoma, which accounts for 74.6% of skin-cancer related deaths. In 2009 alone, there were 68,720 new cases of melanoma diagnosed. Melanoma develops in the melanocytes, which are the melanin producing cells located in the bottom layer of the skin's epidermis. There are four types of melanoma. They are (a) superficial spreading melanoma, (b) lentigo melanoma, (c) acral lentiguous melanoma, and (d) nodular melanoma. All these types of melanoma begin at the top layer of the skin. The first three could become invasive. However, nodular melanoma is invasive from the beginning. Once the type of melanoma has been established, the degree of severity of the disease is determined. Severity of the disease or “stage” is determined by the thickness, depth of penetration, and degree to which the lesion has spread.

Early diagnosis is the key to the treatment of skin cancer. Melanoma can be cured if diagnosed early and treated when the tumor is thin and has not invaded deeply into the dermis of the skin. However, if a melanoma lesion is not removed at an early stage, the cancerous cells may grow downward invading lymphatic channels and blood vessels, resulting in a serious and possibly lethal clinical problem. Currently, the most widely used test to diagnose melanoma is a subjective ABCDE (asymmetry, border, color, diameter, and elevation) test performed by a dermatologist. However, in order to obtain conclusive proof of the malignancy, the patient has to undergo an invasive biopsy.

A method for determining the prognosis with respect to melanoma involves the measurement of the thickness of a lesion. This thickness is also known as Breslow thickness, named after the physician Alexander Breslow, who in the 1970's observed that as the thickness of a tumor increases, the chance of survival goes down. For example, a subject with a lesion of Breslow thickness of 0.75 mm has a five year survival rate of 97%, whereas a subject with a lesion of Breslow thickness of 8 mm has a five year survival rate of less than 32%.

Imaging technology can be used to view skin abnormalities. The past decade has seen a dramatic improvement in the mid infrared (3-300 μm) imaging technology with novel materials, fabrication, and read out integrated circuits. These improvements have lead to the realization of large format (>16 Megapixels), multicolor and higher operating temperature (HOT) infrared focal plane arrays (FPAs).

### BRIEF DESCRIPTION OF THE DRAWINGS

FIG. 1 shows an example technique using an infrared imaging system and a laser, in accordance with various embodiments.

FIG. 2 shows features of an example embodiment of one or more components of a system to determine an identity of an abnormality on a subject using a response from a temperature stimulus applied to the subject, in accordance with various embodiments.

FIG. 3 shows features of an example embodiment of one or more components of a system including an infrared camera and a source of illumination to determine an identity of an abnormality on a subject using a response from a temperature stimulus applied to the subject, in accordance with various embodiments.

FIGS. 4A-4E depict a finite thickness of skin to which the method of FIG. 1 is applied, in accordance with various embodiments.

FIG. 5 shows an example system constructed to perform non-invasive analysis of a subject, in accordance with various embodiments.

FIG. 6 shows an example system arranged to perform non-invasive analysis of a subject, in accordance with various embodiments.

FIG. 7 shows features of an example method of non-invasive analysis of a subject, in accordance with various embodiments.

FIG. 8 depicts a block diagram of features of an example system arranged to conduct non-invasive techniques on a subject, in accordance with various embodiments.

### DETAILED DESCRIPTION

The following detailed description refers to the accompanying drawings that show, by way of illustration and not limitation, various embodiments in which the invention may be practiced. These embodiments are described in sufficient detail to enable those skilled in the art to practice these and other embodiments. Other embodiments may be utilized, and structural, logical, and electrical changes may be made to these embodiments. The various embodiments are not necessarily mutually exclusive, as some embodiments can be combined with one or more other embodiments to form new embodiments. The following detailed description is, therefore, not to be taken in a limiting sense.

In various embodiments, examination of an abnormality on a subject can be conducted using a temperature stimulus applied to the subject, which provides a non-invasive analysis technique. The technique can include applying cold temperature stimuli and hot temperature stimuli. The non-invasive technique is not limited to applying cold temperature stimuli and hot temperature stimuli to change the temperature of a portion of the subject from its ambient temperature. Depending on the application, the cold temperature stimuli can be realized as maintaining an initial temperature of a portion of the subject at its ambient temperature with the hot temperature stimuli being stimuli that supplies sufficient energy to the portion to affect responses such that the portion emits detectable radiation different from the emissions at ambient temperature. In an embodiment, a non-invasive infrared imaging technique can be used to observe the temporal response of a lesion to temperature stimuli to form a basis for evaluating this abnormality on a subject. A technique, which includes applying temperature stimuli and detecting responses to the applied temperature stimuli, provides a non-invasive technique that can be used to identify an abnormality on a subject and/or characteristics of the abnormality. In an embodiment, a non-invasive transient infrared imaging technique can be used to observe the temporal response of a lesion to temperature stimuli to form a basis for determining characteristics correlated to the lesion. Such characteristics can include, but is not limited to, the thickness of the lesion and severity of a disease for which the lesion is a manifestation.

In various embodiments, a method comprises using active and/or passive infrared imaging techniques to non-invasively obtain a three-dimensional (3D) image of an abnormality. This technique may be referred to as “SKI-Scan”. The method may be applied to generate various parameters. For example, the method may be used to obtain the Breslow thickness of a suspected lesion.

In various embodiments, a method comprises examining an abnormality on a subject using a temperature stimulus applied to the subject. In various embodiments, an apparatus comprises one or more components to examine an abnormality on a subject using a temperature stimulus applied to the subject. In various embodiments, a machine-readable storage device having executable instructions stored thereon, which when executed, causes a machine to perform operations comprising examining an abnormality on a subject using a temperature stimulus applied to the subject. Herein, a machine-readable storage device is a physical device that stores data represented by physical structure within the device. Examples of machine-readable storage devices includes, but it not limited to, read only memory (ROM), random access memory (RAM), a magnetic disk storage device, an optical storage device, a flash memory, and other electronic, magnetic, and/or optical memory devices.

In various embodiments, a method comprises determining an identity of an abnormality on a subject or a nature of the abnormality on a subject, using a response from a temperature stimulus applied to the subject. In various embodiments, an apparatus comprises one or more components to determine an identity of an abnormality on a subject or a nature of the abnormality on a subject, using a response from a temperature stimulus applied to the subject. In various embodiments, a machine-readable storage device having executable instructions stored thereon, which when executed, causes a machine to perform operations comprising: determining an identity of an abnormality on a subject or a nature of the abnormality on a subject, using a response from a temperature stimulus applied to the subject.

In past studies, it has been shown that the temperature profile that is obtained using infrared imaging, assuming a value for the emissivity of the skin, is an effective temperature, T_{eff}. T_{eff} is greater than the skin temperature, Tₛ, which is usually 32° C., but is lower than the blood temperature, T_{b}, which is usually 37° C. T_{eff} was determined to be given by

$${T}_{eff} = \frac{{T}_{s}}{n} ( \sqrt{\frac{α}{n β}} - 1 ) \qquad ( Eq . 1 )$$

where α is the absorption coefficient of the skin, β is the coefficient that describes the temperature variation into the epidermis (T=Tₛexp(βx)), and n=C₂/λT, where C₂ is the Dreyfus constant with λ being the wavelength of the emitted electromagnetic radiation. It has also been determined that, if the temperature Tₛ is used instead of T_{eff} to fit the emission from the skin, an anomalously large value of the emissivity of the skin is obtained. It has been concluded that, in the case of the skin temperature Tₛ=32° C. and the blood temperature T_{b}=37° C., T_{eff} is determined to be 34.5° C. Thus, the thickness of the skin that emits the infrared radiation is at least equal to the depth of the blood veins, which is about 20-30 mm.

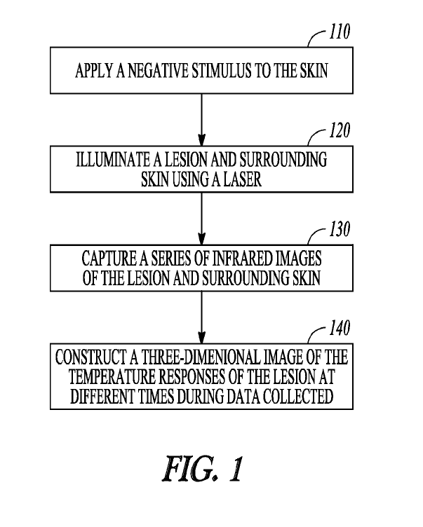

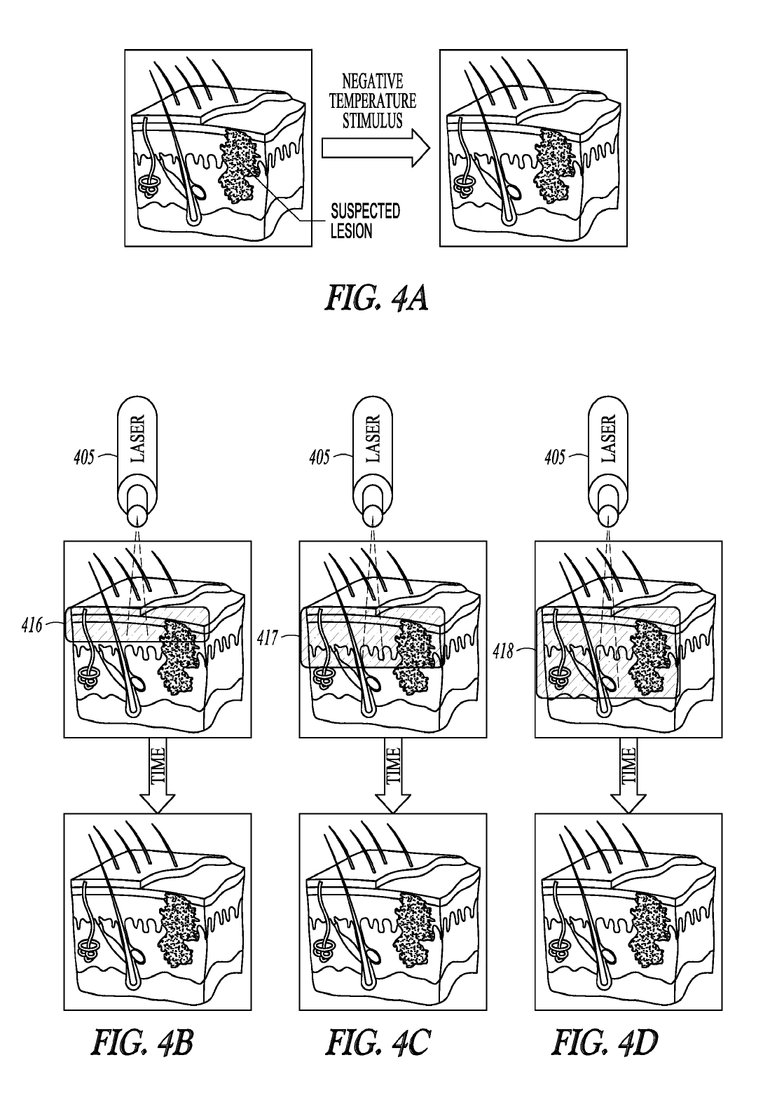

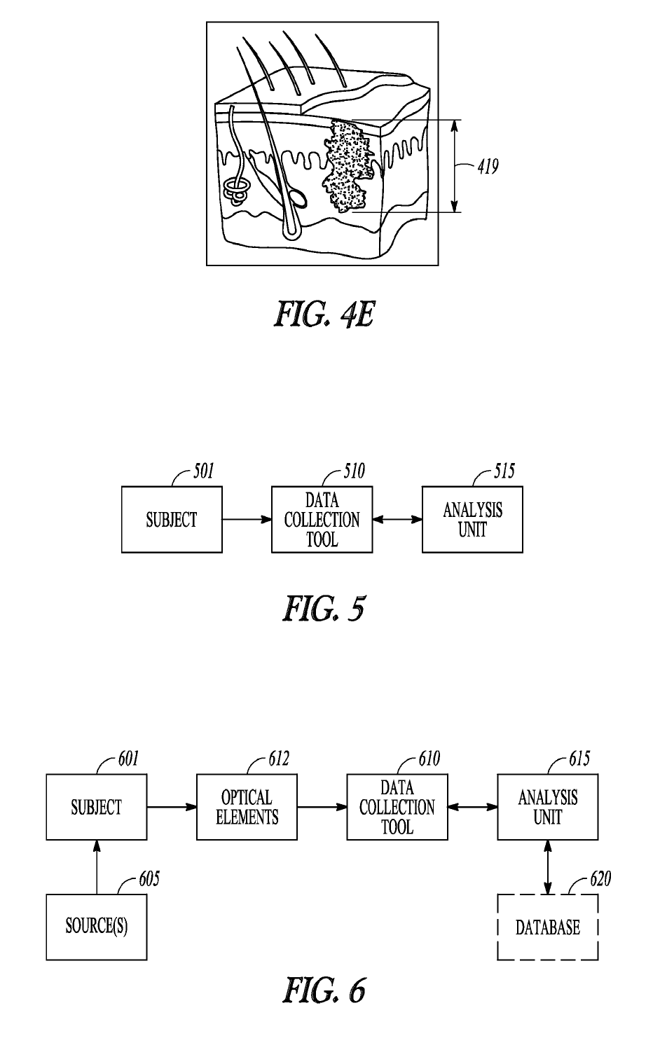

The SKI-Scan technique exploits the fact that a finite thickness of the skin emits the infrared radiation. An embodiment of a SKI-scan technique is shown in FIG. 1. At 110, a negative (cold) temperature stimulus is applied to an entire portion of skin, which is depicted in FIG. 4A. The negative temperature stimulus can be applied using a cold gel pack, for instance. This decreases the temperature of the top 30 mm of the skin to the temperature, T_{gel-pack}, of the gel-back. In other embodiments, the temperature decrease is not limited to the top 30 mm. Other mechanisms may be used to decrease the temperature of the skin.

At 120, a lesion and the surrounding skin in the portion of skin are illuminated using a laser, which is depicted as laser 405 in FIG. 4B. This illumination provides a positive (hot) temperature stimulus at a certain depth, depth 416 shown in FIG. 4B. The illumination can be realized by an infrared laser, for example.

At 130, a series of infrared images of the lesion and the surrounding skin are captured. The infrared images can be captured for 300 seconds as the skin warms up. Other capture times may be used. This procedure can be repeated again by increasing the depth of the positive (hot) temperature stimulus, which depths are depicted as depth 417 in FIG. 4C and as depth 418 in FIG. 4D. The increase in depth can be obtained by varying the intensity of the applied positive stimuli, for example, by varying the power level of the laser.

At 140, using the data collected from these various processes, a 3D image of the temperature responses of the lesion at different times can be constructed, which is depicted in FIG. 4E. The processing of the set of images acquired at the different depths provides a mechanism to capture data corresponding to the entire lesion. From the depth of the transient temperature response, the Breslow thickness, thickness 419 shown in FIG. 4E, can be extracted.

Parameters associated with laser illumination of a lesion and surrounding skin can be varied to examine the lesion. For example, the wavelength of the incident illumination can be changed. Changing the wavelength can be used to change the depth from the surface of the skin that energy is absorbed for heating the region absorbing the radiation. Imaging can be taken with a given distance from the surface exposed to laser illumination. With the wavelength changed, another set of images can be acquired correlated to the depth provided by the changed wavelength. At each distance from the skin surface, the amount of stimulation can be increased by increasing the power of the incident illumination. Alternatively, the amount of stimulation can be decreased by decreasing the power of the incident illumination. In addition, increases or decreases in the amount of temperature stimulation can be realized by changes in the duration of the incident laser illumination. Noting that response to stimuli is different for abnormal cells as compared to normal cells, changing the angle of the incident illumination of laser energy can be used to determine locations in the skin that delineate normal cells from abnormal cells. Changing one or more of the wavelength of incident laser illumination, the power of the incident laser illumination, or the angle of the incident laser illumination in various permutations provides data such that a three-dimensional shape of the lesion can be obtained. In various embodiments, a source of electromagnetic radiation, other than a laser, may be used with the implementation of appropriate optics to controllably direct the radiation to desired locations within the skin.

In various embodiments, a non-invasive transient infrared imaging technique can be used to observe the temporal response of a lesion to a temperature stimulus. The change in the local temperature of the suspected lesion and the surrounding skin can be captured with an infrared camera, in response to a positive or negative temperature stimulus (using a warm or cold gel pack, for instance). Methods and apparatus can be structured based on the transient response of the malignant cells being different compared with the surrounding normal cells. Such methods and apparatus can form a semi-quantitative basis for determining the severity of the disease. For example, methods and apparatus can form a semi-quantitative basis for determining the 3D shape of the abnormality thereby providing an estimate for the severity of a disease associated with the abnormality. If a patient wants confirmation about the malignancy of a particular lesion, various embodiments can provide semi-quantitative data, which can help in determining the nature of the lesion.

In various embodiments, the identified lesion can first be imparted a positive (hot) or negative (cold) temperature stimulus. A negative (cold) temperature stimulus can include, but is not limited to, a cold solid, a cold liquid, a cold gas, or other mechanisms to controllably decrease the temperature. A positive (hot) temperature stimulus can include, but is not limited to, a heated solid, a heated liquid, a heated gas, exposure to electromagnetic radiation such as, but not limited to, radiation from a laser, or other mechanisms to controllably increase the temperature. The use of a laser as a stimulation source can include the use of an infrared laser.

The temporal response of the identified lesion to the temperature stimulus can be captured in a series of infrared images or movies with a broadband infrared camera. In an embodiment, the infrared wavelength of the electromagnetic wave may be 3-300 microns. The infrared camera can be made from a variety of semiconductors including, but not limited to, indium antimonide (InSb), mercury cadmium telluride (MCT), indium gallium arsenide (InGaAs), quantum well infrared photodetectors (QWIP), quantum dot infrared photodetectors (QDIP), type I superlattice detectors, and type II superlattice detectors. A reference marker can be placed in the imaged area and the spatial coordinates of the marker can be used to correct for the voluntary or involuntary movement of the lesion.

The temporal response of the identified lesion to the temperature stimulus can be captured in a series of infrared images or movies with a combination of spectral filters placed in front of an infrared camera. These spectral filters can be lowpass, highpass, bandpass, or notch filters. In an embodiment, the spectral width of the bandpass filters can be from 0.05-100 microns. The infrared camera can be made from a variety of semiconductors including, but not limited to, indium antimonide (InSb), mercury cadmium telluride (MCT), indium gallium arsenide (InGaAs), quantum well infrared photodetectors (QWIP), quantum dot infrared photodetectors (QDIP), type I superlattice detectors, and type II superlattice detectors. A reference marker can be placed in the imaged area and the spatial coordinates of the marker can be used to correct for the voluntary or involuntary movement of the lesion.

The temporal response of the identified lesion to the temperature stimulus can be captured in a series of infrared images or movies with a combination of polarizers placed in front of an infrared camera. The angle of these polarizers can be varied continuously from 0 degrees to 360 degrees. The infrared camera can be made from a variety of semiconductors including, but not limited to, indium antimonide (InSb), mercury cadmium telluride (MCT), indium gallium arsenide (InGaAs), quantum well infrared photodetectors (QWIP), quantum dot infrared photodetectors (QDIP), type I superlattice detectors, and type II superlattice detectors. A reference marker can be placed in the imaged area and the spatial coordinates of the marker can be used to correct for the voluntary or involuntary movement of the lesion.

The temporal response of the identified lesion to the temperature stimulus can be captured in a series of infrared images or movies with a combination of neutral density filters placed in front of an infrared camera. These neutral density filters can be used to change the dynamic range of the infrared image. The infrared camera can be made from a variety of semiconductors including, but not limited to, indium antimonide (InSb), mercury cadmium telluride (MCT), indium gallium arsenide (InGaAs), quantum well infrared photodetectors (QWIP), quantum dot infrared photodetectors (QDIP), type I superlattice detectors, and type II superlattice detectors. A reference marker can be placed in the imaged area and the spatial coordinates of the marker can be used to correct for the voluntary or involuntary movement of the lesion. Full body scans using infrared imaging to monitor any changes in the skin lesions can also be used in a variety of these techniques.

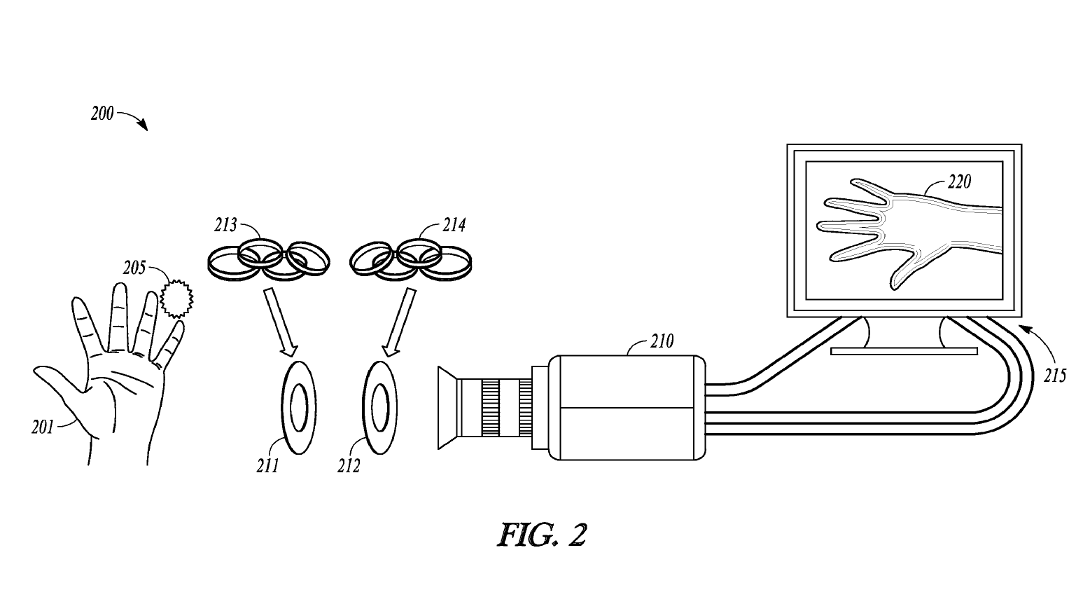

FIG. 2 shows features of an example embodiment of one or more components of a system 200 to determine an identity of an abnormality on a subject using a response from a temperature stimulus applied to the subject. FIG. 2 also illustrates an example of an embodiment of a method in which skin area 201 is imaged and the data from the imaging analyzed. Other body regions can be analyzed used the apparatus and methods discussed herein. System 200 includes a spectral filter 211 or a set 213 of spectral filters, a polarizer filter or analyzer filter 212 or a set 214 of polarizer and analyzer filters, an infrared camera 210, hardware 215 including data acquisition, processing, and analysis components, and software 220 including algorithms directed to data acquisition, processing, and analysis. Herein, an algorithm is a sequence of steps leading to a desired result and software is one or more sets of instructions in the form of physical structure in a device that can be executed under control of a control unit such as a processor. Source 205 operates as an object or instrument to impart a temperature stimulus to the suspected area. Source 205 can be realized as one or more sources to provide positive stimuli and negative stimuli.

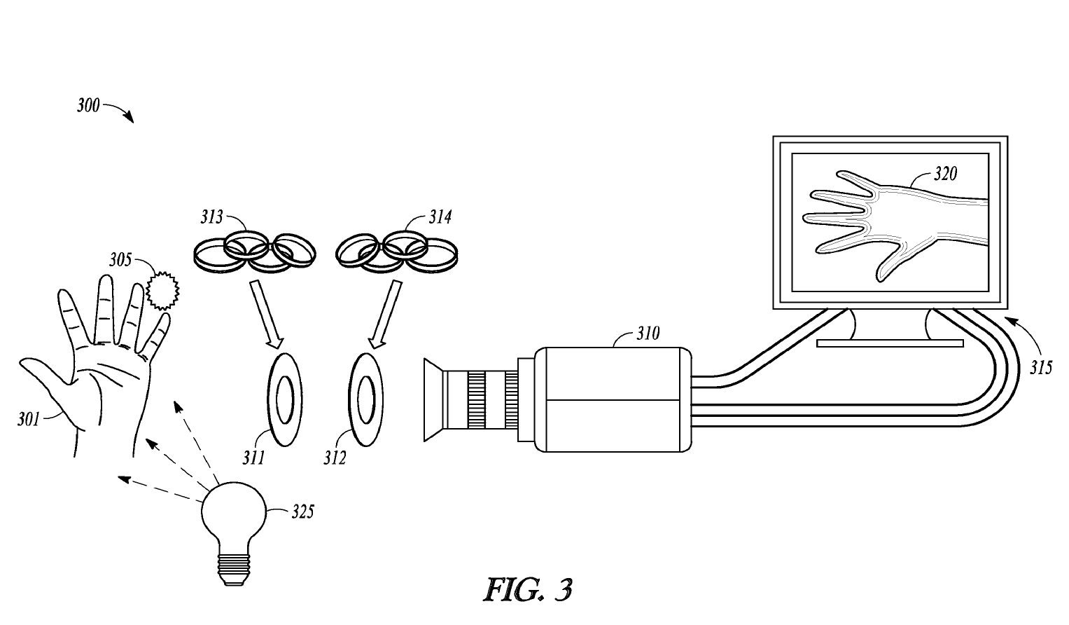

FIG. 3 shows features of an example embodiment of one or more components of a system 300 to determine an identity of an abnormality on a subject using a response from a temperature stimulus applied to the subject. FIG. 3 also illustrates an example of an embodiment of a method in which skin area 301 is imaged and the data from the imaging analyzed. Other body regions can be analyzed used the apparatus and methods discussed herein. System 300 can be constructed similar or identical to system 200 of FIG. 2 with the addition of another temperature source 325. System 300 includes a spectral filter 311 or a set 313 of spectral filters, a polarizer filter or analyzer filter 312 or a set 314 of polarizer and analyzer filters, an infrared camera 310, hardware 315 including data acquisition, processing, and analysis components, and software 320 including algorithms directed to data acquisition, processing, and analysis. Source 305 operates as an object or instrument to impart a temperature stimulus to the suspected area. Source 305 can be realized as one or more sources to provide positive stimuli and negative stimuli. Source 325 can be used as a source of infrared radiation to illuminate region 301. Source 325 can be realized by a laser.

Each of the combination of 215 and 220 and the combination of 315 and 320 provides an analysis unit. The analysis unit can include a database in which characteristics of abnormalities can be stored. These characteristics can be used in a comparison process with measurements acquired using infrared camera 210 (or infrared camera 310) or other data collection tools that can capture electromagnetic radiation from a subject. In addition, the analysis unit can collect data on the abnormality on a subject over time and provide a time-based analysis including the identity of the abnormality, a diagnosis, and a prognosis. The database can store information regarding normal conditions of a subject. Such conditions can be acquired by a full body scan of the subject. A full body scan can also provide a base line for the subject that can be stored in the database. The base line can be obtained before an abnormality appears on the subject.

In various embodiments, a detector can capture transient responses from malignant cells subjected to a temperature probe. The captured responses result from malignant cells having increased metabolic activity relative to normal cells, leading to a higher differential temperature in the measurement. An example of a detector that can be implemented includes a high performance quantum dot camera capable of measuring temperature changes less than 50 mK. As noted, the malignant cells are expected to have an increased metabolic activity, which leads to a change in the local temperature and response to a temperature stimulus. In addition, using spectral filters, polarimetric analyzers, and active illuminators, such as lasers and light emitting diodes, the absolute temperature, morphology and depth of the suspected lesions can be obtained.

Using infrared imaging, one can interpret subcutaneous processes from the cutaneous temperature distribution. Since the emission coefficient of the human skin can be taken as E=0.98±0.01 for λ>2 μm, such an approach can provide the value of the temperature. However if an anomalous region (such as a lesion) has a different emissivity, the temperature cannot be estimated as the problem is ill-defined. To address this problem, measurements under different wavelengths can be made to provide additional equations. The thermographic paradigm holds for near-to-skin processes, since the human core temperature is held constant for depths larger than 20 mm. Consequently, medical diagnosis based on infrared (IR) imaging can be expected to yield results in processes that are close to the skin surface such as pigmented lesions. Using Planck's law, the spectral radiance of electromagnetic radiation emitted in the normal direction from a grey body with emissivity, ε, at a temperature T is given by

$$ρ ( v ) d v = ɛ \frac{2 h {v}^{1}}{{c}^{2}} \frac{1}{{e}^{\frac{h v}{kT}} - 1} d v \qquad Eq . 2$$

where c=speed of light in vacuum, h=Planck's constant, and ν is the frequency of the emitted radiation. The local temperature of a suspected skin lesion can be obtained using a high performance infrared camera if the emissivity of the anomaly and the skin can be measured or estimated.

The transient response of the lesion can be defined with a positive and negative temperature stimulus using a broadband quantum dot infrared camera. The lesion can be imparted a fixed positive (hot) or a negative (cold) temperature stimulus and the temporal response of the subjected area can be monitored using a high performance quantum dot (QD) camera that is capable of measuring a temperature change <50 mK. The spectral content of the transient response of the lesion with a positive and negative temperature stimulus can be evaluated using a spectrally filtered quantum dot infrared camera. Spectral filters can be placed in front of the QD camera to extract spectral and spatial information from the transient response. Obtaining the absolute temperature of the subjected area is enabled by the multispectral imagery. The polarization content of the transient response of the lesion with a positive and negative temperature stimulus can be delineated using a wire grid polarizer coupled with a quantum dot infrared camera. Wire grid polarizer filters and analyzer filters can be placed in front of the QD camera to extract a degree of polarization (DOP) from the transient response, which can be used to obtain the morphology of the malignant and benign cells. Cancer cells are expected to be more spherical than normal cells and this can be captured from the change in their emission with change in the polarization. The data can be corrected for involuntary motion using a reference marker.

In other embodiments, there are provided a device, a method, and a machine readable device as set out below in which various devices, methods, and machine readable devices can be realized in combinations and/or permutations of the devices, methods, and machine readable devices set out below. A first method of non-invasive diagnosis comprises using transient infrared imaging, wherein a lesion is imparted with a positive or negative temperature stimulus, followed by a second positive or negative temperature stimulus at various depths, the temperature change of the lesion and surrounding skin is captured by an infrared camera, and the resultant data is used to identify the 3D structure of the lesion.

In other embodiments, there are provided a device, a method, and a machine readable device as set out below in which various devices, methods, and a machine readable devices can be realized in combinations and/or permutations of the devices, methods, and a machine readable devices set out below. A second method of non-invasive diagnosis comprises using transient infrared imaging, wherein a lesion is imparted with a positive or negative temperature stimulus, the temperature change of the lesion and surrounding skin is captured by an infrared camera and the resultant data is used to identify the nature of lesion. A further embodiment of the second method includes where the lesion imparted with a positive or negative temperature stimulus is followed by a second positive or negative temperature stimulus at various depths, and the resultant data is used to identify the 3D structure of the lesion. A further embodiment of the second method includes the data collected corrected for voluntary or involuntary movement of the lesion using a reference marker.

A further embodiment of the second method includes the method applied where the lesion can be a result of cancer. The lesion can be a result of skin cancer. The lesion can be basal cell carcinoma or squamous cell carcinoma. The lesion can be a melanoma.

A further embodiment of the second method includes where the negative (cold) temperature stimulus can include, but is not limited to, a cold solid, cold liquid, or cold gas. The negative (cold) temperature stimulus can be induced by a laser pulse or set of pulses or continuous wave radiation. The positive (hot) temperature stimulus can be induced by a laser pulse or set of pulses or continuous wave radiation. The positive (hot) temperature stimulus can include, but is not limited to, a heated solid, heated liquid, or heated gas.

A further embodiment of the second method includes a method where the infrared camera can be made from a variety of semiconductors including, but not limited to, indium antimonide (InSb), mercury cadmium telluride (MCT), indium gallium arsenide (InGaAs), quantum well infrared photodetectors (QWIP), quantum dot infrared photodetectors (QDIP), type I superlattice detectors, and type II superlattice detectors.

A further embodiment of the second method includes a method where the temporal response of the identified lesion to the temperature stimulus can be captured in a series of infrared images or movies with a broadband infrared camera. The infrared wavelength of the electromagnetic wave may be between 3-300 microns.

A further embodiment of the second method includes a method where the temporal response of the identified lesion to the temperature stimulus can be captured in a series of infrared images or movies with a combination of spectral filters placed in front of an infrared camera. These spectral filters can be lowpass, highpass, bandpass, or notch filters. The spectral width of the bandpass filters can be from 0.05-100 microns.

A further embodiment of the second method includes a method where the temporal response of the identified lesion to the temperature stimulus can be captured in a series of infrared images or movies with a combination of polarizers placed in front of an infrared camera. The angle of these polarizers can be varied continuously from 0 degrees to 360 degrees.

A further embodiment of the second method includes a method where the temporal response of the identified lesion to the temperature stimulus can be captured in a series of infrared images or movies with a combination of neutral density filters placed in front of an infrared camera. These neutral density filters can be used to change the dynamic range of the infrared image.

A further embodiment of the second method includes a method where content of the infrared camera can be changed in a pixel or subset of pixels. The polarization content of the infrared camera can be changed in a pixel or subset of pixels. The spectral content of the infrared camera can be changed in a pixel or subset of pixels. The dynamic range of the infrared camera can be changed in a pixel or subset of pixels. The relative phase of the pixels or subset of pixels of the infrared camera can be changed.

A further embodiment of the second method includes a method where algorithms in memory devices of an analysis unit under control of one or more processors can be used to extract the difference between the quantitative and qualitative response of the lesion and the normal cells. These differences may be used with data in a database, which may include a full body scan.

A third method of non-invasive diagnosis comprises using transient infrared imaging, wherein a lesion is imparted with a positive or negative temperature stimulus, a source of electromagnetic radiation is used to illuminate the lesion and surrounding region, the associated change of the lesion and surrounding skin is captured by an infrared camera, and the resultant data is used to identify the nature of lesion. A further embodiment of the third method includes a method where the associated change of the lesion and surrounding skin is captured at various depths by the infrared camera. A further embodiment of the third method includes a method where the data collected is corrected for voluntary or involuntary movement of the lesion using a reference marker.

A further embodiment of the third method includes the method applied where the lesion can be a result of cancer. The lesion can be a result of skin cancer. The lesion can be basal cell carcinoma or squamous cell carcinoma. The lesion can be a melanoma.

A further embodiment of the third method includes where the negative (cold) temperature stimulus can include, but is not limited to, a cold solid, cold liquid, or cold gas. The negative (cold) temperature stimulus can be induced by a laser pulse or set of pulses or continuous wave radiation. The positive (hot) temperature stimulus can be induced by a laser pulse or set of pulses or continuous wave radiation. The positive (hot) temperature stimulus can include, but is not limited to, a heated solid, heated liquid, or heated gas.

A further embodiment of the third method includes a method where the infrared camera can be made from a variety of semiconductors including, but not limited to, indium antimonide (InSb), mercury cadmium telluride (MCT), indium gallium arsenide (InGaAs), quantum well infrared photodetectors (QWIP), quantum dot infrared photodetectors (QDIP), type I superlattice detectors, and type II superlattice detectors.

A further embodiment of the third method includes a method where the temporal response of the identified lesion to the temperature stimulus can be captured in a series of infrared images or movies with a broadband infrared camera. The infrared wavelength of the electromagnetic wave may be between 3-300 microns.

A further embodiment of the third method includes a method where the temporal response of the identified lesion to the temperature stimulus can be captured in a series of infrared images or movies with a combination of spectral filters placed in front of an infrared camera. These spectral filters can be lowpass, highpass, bandpass, or notch filters. The spectral width of the bandpass filters can be from 0.05-100 microns.

A further embodiment of the third method includes a method where the temporal response of the identified lesion to the temperature stimulus can be captured in a series of infrared images or movies with a combination of polarizers placed in front of an infrared camera. The angle of these polarizers can be varied continuously from 0 degrees to 360 degrees.

A further embodiment of the third method includes a method where the temporal response of the identified lesion to the temperature stimulus can be captured in a series of infrared images or movies with a combination of neutral density filters placed in front of an infrared camera. These neutral density filters can be used to change the dynamic range of the infrared image.

A further embodiment of the third method includes a method where content of the infrared camera can be changed in a pixel or subset of pixels. The polarization content of the infrared camera can be changed in a pixel or subset of pixels. The spectral content of the infrared camera can be changed in a pixel or subset of pixels. The dynamic range of the infrared camera can be changed in a pixel or subset of pixels. The relative phase of the pixels or subset of pixels of the infrared camera can be changed.

A further embodiment of the third method includes a method where algorithms in memory devices of an analysis unit under control of one or more processors can be used to extract the difference between the quantitative and qualitative response of the lesion and the normal cells. These differences may be used with data in a database, which may include a full body scan.

A further embodiment of the third method includes a method where the source of electromagnetic radiation is a laser. The source of electromagnetic radiation can be a light emitting diode. The source of electromagnetic radiation can be a broad band source. The source of electromagnetic radiation can be a narrow band source.

A further embodiment of the third method includes a method where the associated change can result in information about the depth of the lesion and the surrounding skin. The associated change can be a change in reflectance of the incident radiation. The associated change can be a change in spectral content of the incident radiation. The associated change can be a change in polarization of the light of the incident radiation. The associated change can be a change in transmission of the incident radiation. The associated change can be a change in absorption of the incident radiation. The associated change can be a change in amplitude of the incident radiation. The associated change can be a change in phase of the incident radiation. The associated change can be a change in spatial content of the incident radiation.

A further embodiment of the third method includes a method where the wavelength of the electromagnetic radiation of the incident illumination is used to illuminate is changed. The angle of the electromagnetic radiation of the incident illumination can be changed. The power of the electromagnetic radiation of the incident illumination can be changed. The duration of the electromagnetic radiation of the incident illumination can be changed.

A further embodiment of the third method includes a method where a three-dimensional shape of the lesion can be obtained. The three-dimensional shape of the lesion can be analyzed to determine other characteristics of the lesion. These characteristics can be applied to further diagnosis and prognosis.

In various embodiments, apparatus comprise one or more components arranged to perform operations of one or more of example methods one, two, three, and their embodiments above or various combinations thereof. Comparisons of collected data can be performed by the apparatus using data and/or standards accessible by the components of the apparatus. The data can include versions of data collected from an abnormality over time. The data can be from a database of characteristics of different abnormalities that may occur on a subject.

In various embodiments, a machine-readable storage device having executable instructions stored thereon, which when executed, causes a machine to perform operations comprising one or more of example methods one, two, three, and their embodiments above or various combinations thereof. The machine-readable storage device can be any data storage device. For example, the machine-readable storage device can be a storage device for a computer in which the instructions can be executed using a controller such as one or more processors.

FIG. 5 shows an example embodiment of a system 500 constructed to perform non-invasive analysis of a subject 501. System 500 includes a data collection tool 510 and an analysis unit 515 coupled to data collection tool 510. Data collection tool 510 is operable to capture electromagnetic radiation from a subject. Analysis unit 515 is arranged to identify an abnormality and/or a characteristic of the abnormality in a portion of the subject using images of the portion captured by data collection tool 510 at different times during application of a set of temperature stimuli to the portion. System 500 can be arranged to conduct non-invasive operations for analysis in accordance with the methods taught herein.

FIG. 6 shows an example of a system 600 having components, in addition to the components of system 500 of FIG. 5, to perform non-invasive analysis of a subject 601. System 600 includes one or more sources 605 to generate the temperature stimuli directed to the subject, whose responses can be analyzed by an analysis unit 615 coupled to a data collection tool 610. The one or more sources 605 can include one or more of a source of pulses of electromagnetic radiation, a source of continuous electromagnetic radiation, a pulse laser device, a continuous laser device, a source of hot stimuli including one or more of a heated solid, a heated liquid, or a heated gas, a source of cold stimuli including one or more of a cold solid, a solid liquid, or a cold gas.

Data collection tool 610 can include an infrared camera. The infrared camera can be capable of providing a measure of emissivity and/or temperature. The infrared camera can include a photo responsive structure having one or more of a indium antimonide (InSb) based structure, a mercury cadmium telluride (MCT) based structure, an indium gallium arsenide (InGaAs) based structure, a quantum well infrared photodetector (QWIP), a quantum dot infrared photodetectors (QDIP), a type I superlattice detector, or a type II superlattice detector. The infrared camera can include a broadband infrared camera.

Data collection tool 610 can include an infrared camera with one or more optical elements 612 disposed in front of the infrared camera such that in operation optical elements 612 are between the infrared camera and the subject. Optical elements 612 include one or more of a spectral filter, a polarizer, or a neutral density filter. The spectral filter can include a lowpass filter, a highpass filter, a bandpass filter, or a notch filter. The polarizer used can have an angle continuously variable from 0 degrees to 360 degrees. The neutral density filter can be operable to change a dynamic range of an infrared image being captured by the infrared camera. Optical elements 612 can include components arranged similar or identical to the various optical components associated with FIGS. 2 and 3.

Analysis unit 615 can include one or more processors and one or more memory devices having instructions stored thereon such that the analysis unit is operable to extract differences between quantitative and qualitative responses of a lesion and normal cells within the portion of the subject to which stimuli are applied. System 600 may include a database 620 accessible to analysis unit 615. Database 620 can be arranged to store data corresponding to a full body scan of the subject. Database 620 may be integrated with analysis unit 615, separated from analysis unit 615 and communicatively coupleable to analysis unit 615, or combinations of integrated components and separate components from analysis unit 615. System 600 can be arranged to conduct non-invasive operations for analysis in accordance with the methods taught herein.

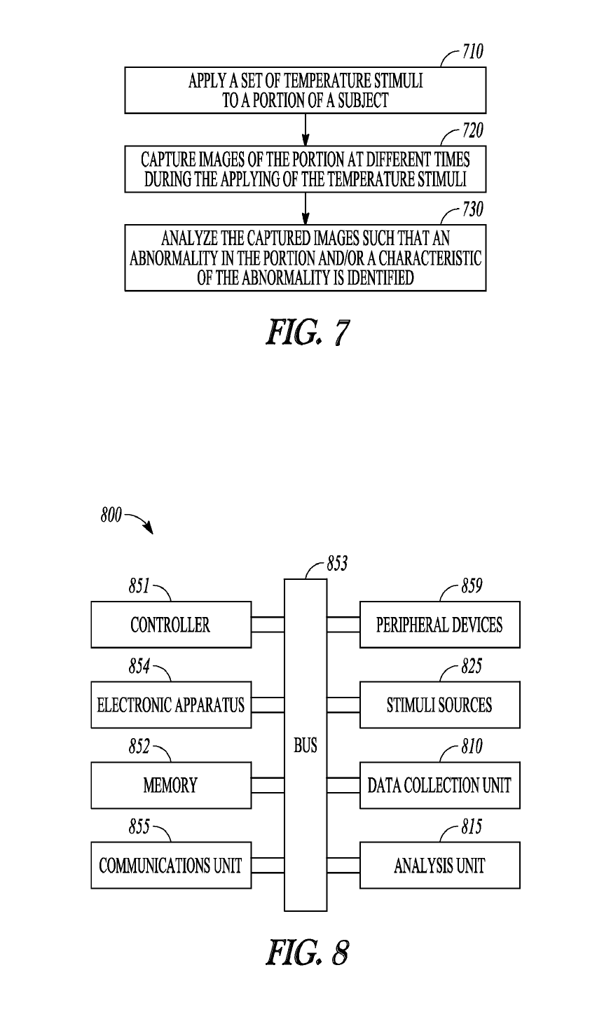

FIG. 7 shows features of an embodiment of a method of non-invasive analysis of a subject. At 710, a set of temperature stimuli are applied to a portion of the subject. Applying a set of temperature stimuli can include applying a cold temperature stimulus followed by a hot temperature stimulus. Applying a set of temperature stimuli can include applying a cold temperature stimulus using one or more of a cold solid, cold liquid, or cold gas and applying a hot temperature stimulus using one or more of a hot solid, hot liquid, hot gas, or exposure to electromagnetic radiation. Exposure to electromagnetic radiation can include laser illumination of the portion.

At 720, images of the portion are captured at different times during the applying of the temperature stimuli. Capturing the images can include using a data collection tool operable to capture electromagnetic radiation from the subject. The non-invasive process can include adjusting the set of temperature stimuli to capture images corresponding to different depths from a surface of the subject. Adjusting the set of temperature stimuli can include changing parameters of laser illumination used as a source of temperature stimulation of the portion. Changing parameters of laser illumination can include changing a wavelength of the laser illumination or changing power of the laser illumination or changing duration of the laser illumination or changing the angle of the laser illumination incident on the portion or a combination of changing the wavelength, the power, the duration, and the angle.

At 730, the captured images are analyzed such that an abnormality in the portion and/or a characteristic of the abnormality is identified. The analysis can be performed under the control of a processing unit. Spatial coordinates of a reference marker can be used to correct for voluntary or involuntary movement of the abnormality. Analyzing the captured images can include analyzing captured images corresponding to the different depths, which can include comparing responses from the abnormality with responses from regions in the portion surrounding the abnormality that are different from the abnormality. The processing unit can control constructing a three dimensional image of responses of the portion to the set of temperature stimuli. The abnormality may include a lesion. With respect to the lesion, the analysis may include extracting differences between responses, associated with emissivity and/or temperature, of the lesion in the portion to the temperature stimuli and responses of normal cells in the portion to the temperature stimuli. The analysis may include extracting a Breslow thickness of the lesion from the captured images corresponding to the different depths. The analysis may include comparing data from the captured images with a base line of a full body scan of the subject. The base line can be stored in a database. The analysis may include collecting versions of data from the abnormality over time in a database.

Various processes, as taught herein, can use one or more machine-readable storage device having executable instructions stored thereon, which instructions when executed, causes a machine to perform operations comprising the selected process. In addition, various processes of non-invasive analysis can be implemented using apparatus, as taught herein, similar to or identical to the apparatus associated with FIGS. 1-6, and 8 and combinations and/or permutations thereof.

FIG. 8 depicts a block diagram of features of an example embodiment of a system 800 arranged to conduct non-invasive techniques on a subject. System 800 includes a data collection tool 810 and components to conduct analysis of data acquired by data collection tool 810. Data collection tool 810 and the components to conduct analysis can be structured similar to or identical to a configuration associated with any of FIGS. 1-7.

System 800 can include a controller 851, a memory 852, an electronic apparatus 854, and a communications unit 855. Controller 851, memory 852, and communications unit 855 can be arranged to operate as a processing unit to control management of data collection tool 810 and analysis of data collected by data collection tool 810 and to perform operations on data signals used to control stimuli sources 825 to apply temperature stimuli to the subject. An analysis unit can be distributed among the components of system 800 including electronic apparatus 854. Alternatively, system 800 can include an analysis unit 815 to manage the analysis of data collected.

System 800 can also include a bus 853, where bus 853 provides electrical conductivity among the components of system 800. Bus 853 can include an address bus, a data bus, and a control bus, each may be independently configured. Bus 853 can be realized using a number of different communication mediums that allows for the distribution of components of system 800. Use of bus 853 can be regulated by controller 851.

In various embodiments, peripheral devices 859 can include displays, additional storage memory, and/or other control devices that may operate in conjunction with controller 851 and/or memory 852. In an embodiment, controller 851 can be realized as a processor or a group of processors that may operate independently depending on an assigned function. Peripheral devices 859 can include a display, which may be arranged as a distributed component, that can be used with instructions stored in memory 852 to implement a user interface to manage the operation of data collection tool 810, analysis unit 815, and/or components distributed within system 800. Such a user interface can be operated in conjunction with communications unit 855 and bus 853.

Although specific embodiments have been illustrated and described herein, it will be appreciated by those of ordinary skill in the art that any arrangement that is calculated to achieve the same purpose may be substituted for the specific embodiments shown. Upon studying the disclosure, it will be apparent to those skilled in the art that various modifications and variations can be made in the devices and methods of various embodiments of the invention. Various embodiments can use permutations and/or combinations of embodiments described herein. It is to be understood that the above description is intended to be illustrative, and not restrictive, and that the phraseology or terminology employed herein is for the purpose of description.

1. A system comprising:
a data collection tool operable to capture electromagnetic radiation from a subject; and
an analysis unit coupled to the data collection tool, the analysis unit arranged to identify an abnormality and/or a characteristic of the abnormality in a portion of the subject using images of the portion captured by the data collection tool at different times during application of a set of temperature stimuli to the portion.

2. The system of claim 1, wherein the system includes one or more sources to generate the temperature stimuli.

3. The system of claim 2, wherein the one or more sources includes one or more of a source of pulses of electromagnetic radiation, a source of continuous electromagnetic radiation, a pulse laser device, a continuous laser device, a source of hot stimuli including one or more of a heated solid, a heated liquid, or a heated gas, a source of cold stimuli including one or more of a cold solid, a solid liquid, or a cold gas.

4. The system of claim 1, wherein the data collection tool includes an infrared camera capable of providing a measure of emissivity and/or temperature.

5. The system of claim 4, wherein the infrared camera includes a photo responsive structure having one or more of a indium antimonide (InSb) based structure, a mercury cadmium telluride (MCT) based structure, an indium gallium arsenide (InGaAs) based structure, a quantum well infrared photodetector (QWIP), a quantum dot infrared photodetectors (QDIP), a type I superlattice detector, or a type II superlattice detector.

6. The system of claim 4, wherein the infrared camera includes a broadband infrared camera.

7. The system of claim 1, wherein the data collection tool includes an infrared camera with optical elements disposed in front of the infrared camera such that in operation the optical elements are between the infrared camera and the subject.

8. The system of claim 7, wherein the optical elements include one or more of a spectral filter, a polarizer, or a neutral density filter.

9. The system of claim 8, wherein the spectral filter includes a lowpass filter, a highpass filter, a bandpass fitter, or a notch filter.

10. The system of claim 8, wherein the polarizer has an angle continuously variable from 0 degrees to 360 degrees.

11. The system of claim 8, wherein the neutral density filter is operable to change a dynamic range of an infrared image being captured by the infrared camera.

12. The system of claim 1, wherein the analysis unit includes one or more processors and one or more memory devices having instructions stored thereon such that the analysis unit is operable to extract differences between quantitative and qualitative responses of a lesion and normal cells within the portion.

13. The system of claim 1, wherein the system includes a database accessible to the analysis unit, the database arranged to store data corresponding to a full body scan of the subject.

14. A method comprising:
applying a set of temperature stimuli to a portion of a subject;
capturing images of the portion at different times during the applying of the temperature stimuli, capturing the images including using a data collection tool operable to capture electromagnetic radiation from the subject; and
analyzing, under the control of a processing unit, the captured images such that an abnormality in the portion and/or a characteristic of the abnormality is identified.

15. The method of claim 14, wherein the method includes constructing, under the control of the processing unit, a three dimensional image of responses of the portion to the set of temperature stimuli.

16. The method of claim 14, wherein applying a set of temperature stimuli includes applying a cold temperature stimulus followed by a hot temperature stimulus.

17. The method of claim 14, wherein applying a set of temperature stimuli includes applying a cold temperature stimulus using one or more of a cold solid, cold liquid, or cold gas and applying a hot temperature stimulus using one or more of a hot solid, hot liquid, hot gas, or exposure to electromagnetic radiation.

18. The method of claim 17, wherein exposure to electromagnetic radiation includes laser illumination of the portion.

19. The method of claim 14, wherein the method includes adjusting the set of temperature stimuli to capture images corresponding to different depths from a surface of the subject.

20. The method of claim 19, wherein the abnormality includes a lesion.

21. The method of claim 20, wherein the method includes extracting differences between responses, associated with emissivity and/or temperature, of the lesion in the portion to the temperature stimuli and responses of normal cells in the portion to the temperature stimuli.

22. The method of claim 20, wherein the method includes extracting a Breslow thickness of the lesion from the captured images corresponding to the different depths.

23. The method of claim 19, wherein analyzing the captured images corresponding to the different depths includes comparing responses from the abnormality with responses from regions in the portion surrounding the abnormality that are different from the abnormality.

24. The method of claim 19, wherein adjusting the set of temperature stimuli includes changing parameters of laser illumination used as a source of temperature stimulation of the portion.

25. The method of claim 24, wherein changing parameters of laser illumination includes changing a wavelength of the laser illumination or changing power of the laser illumination or changing duration of the laser illumination or changing the angle of the laser illumination incident on the portion or a combination of changing the wavelength, the power, the duration, and the angle.

26. The method of claim 14, wherein the method includes comparing data from the captured images with a base line of a full body scan of the subject, the base line stored in a database.

27. The method of claim 14, wherein the method includes using spatial coordinates of a reference marker to correct for voluntary or involuntary movement of the abnormality.

28. The method of claim 14, wherein method includes collecting versions of data from the abnormality over time in a database.

29. A machine-readable storage device having executable instructions stored thereon, which instructions when executed, causes a machine to perform operations comprising the method of any of claims 14 to 28.

30. A system comprising:
a data collection tool operable to capture electromagnetic radiation from a subject; and an analysis unit coupled to the data collection tool, the analysis unit arranged with the data collection tool to perform operations of any of claims 14 to 28.

31.-37. (canceled)
